(19) United States
(12) Patent Application Publication
(10) Pub. No.: US 2007/0207169 A1
(43) Pub. Date: Sep. 6, 2007

(54) EARLY CHILDHOOD INFECTION WITH HUMAN HERPES VIRUS TYPE 6 (HHV-6) AS A PROTECTION AGAINST THE DEVELOPMENT OF IGE SENSITIZATION, ATOPIC DISEASE AND ALLERGY IN YOUNG CHILDREN

(76) Inventors: Inger Nordstrom, Goteborg (SE); Kristina Eriksson, Molndal (SE)

Correspondence Address: GAUTHIER & CONNORS, LLP, 225 FRANKLIN STREET, SUITE 2300, BOSTON, MA, 02110, US

(21) Appl. No.: 11/687,853

(22) Filed: Mar. 19, 2007

Related U.S. Application Data
(63) Continuation of application No. PCT/SE05/01357, filed on Sep. 16, 2005.

(30) Foreign Application Priority Data
Sep. 17, 2004 (SE) 0401790-1
Sep. 17, 2004 (SE) 0402268-7

(51) Int. Cl. A61K 39/245

(52) U.S. Cl. 424/231.1

(57) ABSTRACT

The present invention discloses a use of human herpes virus 6 (HHV-6) or derivatives thereof, for the manufacture of a pharmaceutical composition intended to be administered during early childhood, for the prevention of IgE sensitization and allergy development in young children. Examples of viral compositions include, but are not limited to live human herpes virus 6 (HHV-6).

### TECHNICAL FIELD

The invention refers to the use of an early childhood infection with HHV-6 (before 18 months of age) as a protective effect against the development of IgE sensitization, atopy and atopic disease including asthma.

### BACKGROUND OF THE INVENTION

Why the prevalence of allergic diseases has increased so dramatically in the developed world during the last decades remains an enigma. Allergy is much more common in industrialized countries compared to developing countries, which also applies to autoimmune and inflammatory disorders. One theory, commonly referred to as the hygiene hypothesis, postulates that the improved living standard, which in its turn reduces the incidence of childhood infections, might be one important factor. The hygiene hypothesis was initially based on the observation that children that are more consistently exposed to infections, such as children from large families and those attending day care, are statistically less likely to develop atopic disease than e.g. first-born or single children. This has led to the speculation that exposure to microbes in early childhood affords proper maturation of the developing immune system. However, it is not known which types of microbes are important for this to occur. There is an endless variety of bacteria, viruses and parasites, some of which might be important in providing the right type of stimuli to the immune system, others which may be ineffective, or even increase the risk of developing hypersensitivity or inflammation.

Allergy is defined as an enhanced immune reactivity to harmless environmental antigens, so called allergens. In general adults and children without atopy mount a low-grade immunologic response; they produce allergen specific IgG antibodies and in vitro their T cells respond to the allergen with a moderate degree of proliferation and the production of interferon-γ by Th1 cells (infection fighters). By contrast, persons with atopy have an exaggerated response characterized by the production of allergen-specific IgE antibodies; they have elevated serum levels of IgE antibodies and positive reactions to extracts of common allergens on skin-prick tests. In IgE-mediated allergies, the allergic individual mounts an IgE-antibody response to proteins in foodstuffs, pollens, animal dander, etc. The IgE antibodies are produced by plasma cells developed from B cells with specificity for a certain allergen. To become an IgE-producing plasma cell, the B cell must receive help from a T cell which is specific towards the same allergen. T cells from blood from an allergic individual respond to allergens in vitro by inducing cytokines (i.e., interleukin-4, 5 and 13) produced by type Th2 cells which promotes maturation of the B cell into a plasma cell that produces IgE. The balance between allergy promoting Th2 cells and infection fighting Th1 cells is a critical component of our immune system. There are many exceptions to this rule but the immunopathological hallmark of allergic disease is the infiltration of Th2 cells (allergy promoters) in affected tissue. Th2 cells also commonly produce IL-5, a cytokine which promotes maturation of eosinophils in the bone marrow, and activation of such eosinophils which arrive to the tissue cause an allergic reaction to take place. Once IgE antibodies are formed, they attach to mast cells in the tissue, for example around blood vessels and in the respiratory and gastrointestinal tracts. When the allergic individual is exposed to an allergen, e.g. via inhalation or ingestion, minute amounts of intact protein allergen are taken up into the circulation, reach the mast cells and bind to the IgE antibodies. Hereby the mast cell becomes activated and secretes a range of mediators that trigger the allergic reaction, leading to symptoms forming disease entities such as hay fever, asthma, urticaria, atopic eczema and allergic anaphylaxis.

Several common viral infections have been linked to the development of atopy and allergy. The most clear-cut example of a viral infection that might counteract the development of atopy is hepatitis A virus (HAV), which may protect individuals from allergic disease if they carry a particular variant of the gene that encodes the HAV cell-surface receptor TIM-1, [McIntire, J. J., S. E. Umetsu, C. Macaubas, E. G. Hoyte, C. Cinnioglu, L. L. Cavalli-Sforza, G. S. Barsh, J. F. Hallmayer, P. A. Underhill, N. J. Risch, G. J. Freeman, R. H. DeKruyff, and D. T. Umetsu. 2003. Immunology: hepatitis A virus link to atopic disease. Nature 425:576].

Epidemiological studies support this notion as HAV seropositivity is inversely associated with atopy. However, previous exposure to other viruses such as measles, mumps, rubella, chickenpox, cytomegalovirus, and herpes simplex virus type 1, is as common among atopic and non-atopic Italian subjects, [Matricardi, P. M., F. Rosmini, S. Riondino, M. Fortini, L. Ferrigno, M. Rapicetta, and S. Bonini. 2000. Exposure to foodborne and orofecal microbes versus airborne viruses in relation to atopy and allergic asthma: epidemiological study. Bmj 320:412].

There are numerous studies showing that children who develop lower respiratory symptom during infection with respiratory syncytial virus (RSV) in early life are at increased risk for developing asthma-like syndromes later in childhood. However, severe symptoms following RSV infection is more common in children with a strong family history of allergy. Genetic analysis shows that there is an overrepresentation of an IL-4 haplotype that predisposes for asthma in patients with severe RSV infection. It has thus been suggested that RSV bronchiolitis rather reflects the predisposition of the individual for asthma later in life, than being the actual cause of allergic airway disease.

Herpes viruses are part of the normal human flora. Most human herpes virus infections have a high prevalence, and transmission of the most common herpes viruses usually occurs during childhood. All human herpes viruses can establish latency within the infected host and thereby a life-long infection. HHV-6 is often the earliest of the herpes viruses to infect the young child. It is the etiological agent of exanthema subitum, also known as roseola infantum or sixth disease in children, which is a benign childhood disease characterized by high fever for 3-5 days, sometimes followed by a self-limiting rash. HHV-6 is a ubiquitous virus and the primary infection generally occurs during the first year of life. In Sweden, more than 90% of adults are infected, with 60% of children infected before 1 year of age. HHV-6 replicates most efficiently in activated T-cells but the virus has a broad host cell range and infection in vivo occurs in a variety of cells including macrophages, dendritic cells, B-cells and NK (natural killer) cells. The virus is continuously shed in the saliva, which is believed to serve as the main route of transmission. It should be pointed out that complications after primary HHV-6 infection are uncommon and rarely fatal except in immunocompromised individuals.

As discussed above, non-allergic individuals do not mount IgE antibody responses to common environmental antigens, or they may develop a transient and weak IgE response to food antigens which gradually disappears. The propensity to develop allergy is established in the first few years of life (even if the allergy may manifest itself much later), which has led to a number of measures in order to try to prevent allergy development in children. For example, exclusive breast-feeding and avoidance of exposure to allergens have been widely promoted for many years. However, these measures have been completely ineffective, in that only minute amounts of antigen is needed to trigger IgE production. Many infants may, in fact, develop allergies to egg and cow's milk proteins while being exclusively breast-fed. Furthermore, children from families who have avoided pets are no less allergic to cats and dogs than children who have grown up with such pets in the family.

The ineffectiveness of the above measures in reducing allergies has prompted the search for alternative preventive measures. Yoghurts and other traditional fermented food products have been tried both as therapeutic and preventive agents against allergy. Lactobacillus rhamnosus GG was given to children with severe cow's milk allergy and was shown to ameliorate intestinal inflammation and eczema in these patients. Based on these positive effects, Lactobacillus rhamnosus GG was given to mothers during pregnancy and lactation, and to bottle-fed infants in their formula, as a means to prevent development of allergy in their children. Indeed, children who were exposed to these lactobacilli had less eczema by two and four years of age compared to children who were not exposed to these bacteria. However, it is important to note that there was no reduction in IgE levels in children who had been exposed to these lactobacilli during infancy (Kalliomäki et al. Lancet. 2001 Apr. 7; 357(9262):1076-9 and Kalliomäki et al. Lancet. 2003 May 31; 361(9372):1869-71).

The present invention discloses a solution to this problem. By an early childhood exposure to human herpes virus 6 (HHV-6), the development of a Th2 response is down-modulated, thus limiting the maturation of B cells into IgE producing plasma cells. By this pretreatment, production of IgE antibodies will be prevented and allergic reactions are decreased or avoided when the child becomes exposed to potential allergens in the food and environment.

### SUMMARY OF THE PRESENT INVENTION

The present invention discloses a use of human herpes virus 6 (HHV-6) or derivatives thereof, for the manufacture of a pharmaceutical composition intended to be administered during early childhood, for the prevention of IgE sensitization, atopic disease and allergy development in young children. Examples of viral compositions include, but are not limited to live human herpes virus 6 (HHV-6).

In one preferred embodiment of the invention the pharmaceutical composition comprises live human herpes virus 6 (HHV-6).

In one preferred embodiment of the invention the pharmaceutical composition comprises weakened human herpes virus 6 (HHV-6).

In one preferred embodiment of the invention the pharmaceutical composition comprises killed human herpes virus 6 (HHV-6).

In one preferred embodiment of the invention the pharmaceutical composition comprises one or more fragments of human herpes virus 6 (HHV-6).

In one preferred embodiment of the invention the pharmaceutical composition comprises one or more fragments of human herpes virus 6 (HHV-6) synthesized by recombinant technology such as genetic engineering or synthetic chemistry.

In another preferred embodiment of the invention, the pharmaceutical composition comprising the human herpes virus 6 (HHV-6) is administered orally.

In another preferred embodiment of the invention, the pharmaceutical composition comprising the human herpes virus 6 (HHV-6) is administered parenterally.

In another preferred embodiment of the invention, the pharmaceutical composition comprising the human herpes virus 6 (HHV-6) is administered onto a mucous membrane, such as the nasal mucous membrane.

In a further preferred embodiment the pharmaceutical composition comprising the human herpes virus 6 (HHV-6), is administered to a child no later than 3 years after birth, more preferably no later than 2 years after birth, more preferably no later than 18 months after birth, more preferably no later than 12 months after birth, more preferably no later than 6 months after birth, and even more preferably no later than 4 months after birth.

In another preferred embodiment of the invention, the pharmaceutical composition comprising the human herpes virus 6 (HHV-6) down-modulates the allergic response to allergens.

In another preferred embodiment of the invention, the pharmaceutical composition comprising the human herpes virus 6 (HHV-6) inactivates antigen-specific T-cells.

In another preferred embodiment of the invention, the pharmaceutical composition comprising the human herpes virus 6 (HHV-6) promotes T-cells to secrete cytokines that counteract the development of a Th2 response.

In another preferred embodiment of the invention, the pharmaceutical composition comprising the human herpes virus 6 (HHV-6) promotes the production of regulatory T cells.

In another preferred embodiment of the invention, the pharmaceutical composition comprising the human herpes virus 6 (HHV-6) promotes the secretion of a chemokine from T cells binding the chemokine receptor CCR2 to counteract the development of a Th2 response.

In a further preferred embodiment the invention relates to a pharmaceutical composition wherein the composition is present as a liquid formulation.

In a further preferred embodiment the invention relates to a pharmaceutical composition wherein the composition is present as a solid formulation.

In an additional aspect of the invention, the use of the pharmaceutical composition comprising the human herpes virus 6 (HHV-6) provides a method for preventing atopy and atopic disease including asthma in children.

### DETAILED DESCRIPTION OF THE PRESENT INVENTION

According to the present invention an early childhood infection with HHV-6 (before 18 months of age) has a protective effect on the development of IgE sensitization and atopic diseases including asthma. In a cohort of 19 allergic children, i.e. with defined allergen-specific IgE and/or clinically defined allergy and 38 healthy children aged 18 months, it was found that only 16% of the allergic children have antibodies to HHV-6, whereas 53% of the healthy children have HHV-6 antibodies (p<0.01, Fishers exact test). Or, put in another way, only 16% of HHV-6-infected children are allergic at 18 months of age as compared to 47% of HHV-6-seronegative children. No relationship between the development of allergy and antibodies to other members of the human herpes virus family, including cytomegalovirus, Epstein-Barr virus, varicella zoster virus or herpes simplex virus, could be documented. The observed relationship between HHV-6 infection and atopy suggests a strong link between a common and benign infectious childhood disease and the incidence of allergen-specific IgE and clinically defined allergy.

There are several interesting features in the pathogenesis of HHV-6 infection that could possibly contribute to the reduced incidence of atopy in our cohort of HHV-6 infected children. First of all, HHV-6 infects both antigen-presenting cells and T-cells, and the infection could therefore have a strong impact on the differentiation of an ongoing immune response, including the development of atopy. Recent studies suggest that HHV-6-infected dendritic cells have a reduced capacity to process and present foreign antigen and thus to activate antigen-specific T-cells. Our data show that HHV-6-infected plasmacytoid dendritic cells secrete high levels of IFN-α and IP-10, cytokines which both contribute to the deviation of the immune response from Th2 to Th1. In accordance with this, we also found that HHV-6-infected plasmacytoid dendritic cells decrease the production of the Th2 cytokine IL-13 and increased the production of the Th1 cytokine IFN-α in cord-blood T-cells. Thus, we propose that one mechanism whereby HHV-6 protects from IgE sensitization and allergy development is through a direct effect on dendritic cells which in their turn affect the Th1/Th2 balance. The effect of HHV-6-infection of T-cells is more profound. Infected cells often die from apoptosis or secrete cytokines that would counteract the development of a Th2 response. HHV-6 infection might therefore influence their capacity to induce allergen-specific Th1/Th2 balance. HHV-6-infection of blood mononuclear cells promotes the production of cytokines that are linked to regulatory T-cells. It is believed that allergy, autoimmune and inflammatory disorders are prevented by so called regulatory T cells. These cells suppress activation of helper T cells and thereby down-regulate many types of immune responses

Secondly, HHV-6 utilizes CD46, a membrane cofactor protein, as its receptor. CD46 is expressed on all nucleated cells and its main function is to protect our own cells against inappropriate complement activation and deposition on the plasma membranes, especially by the alternative pathway of complement activation. T-cells are the main targets for HHV-6. In vitro studies show that infected T-cells secrete Th1 and inflammatory cytokines, and are prone for apoptosis. It is not known if Th1 and Th2 cells express the HHV-6 receptor CD46 to the same degree and thus have a different susceptibility to HHV-6 infection. Thus, HHV-6-infected cells, which down-modulate CD46 expression, are more prone to be the targets of complement-mediated killing.

CD46 is also involved in IgE production. Cross-linking CD46 on B-cells synergize with IL-4 in promoting IgE class switching. Thus a reduced expression of CD46 on HHV-6 infected B-cells might prevent the production of IgE antibodies. Furthermore, HHV-6 engagement of CD46 on a recently activated T-cell can induce the formation of a subset of regulatory T-cells, thus influencing the differentiation of T-cells into regulatory T-cells.

Thirdly, HHV-6-infected T-cells secrete a HHV-6-encoded chemokine (a protein that regulates the development and migration of various cells) which binds to a specific chemokine receptor, CCR2, on human host cells. CCR2 is expressed on a variety of cells including monocytes, T- and B-lymphocytes, basophils, mast cells and neutrophils. There is a strong link between CCR2 and allergic responses. Mice that lack the CCR2 receptor are more prone to develop harmful Th2 responses, including elevated IgE levels, after exposure to allergens.

This indicates that CCR2 ligation might block the induction of an allergic response by down-modulating Th2 development. In this context, it is interesting to note that CCR2 is also expressed on regulatory T-cells thus the chemokine secreted from HHV-6-infected T-cells can alter the fate of the differentiating T-cell.

Pharmaceutical Compositions

The compounds of the present invention may be isolated in any level of purity by standard methods and purification can be achieved by conventional means known to those skilled in the art, such as distillation, recrystallization and chromatography.

The compounds of the invention may be administered alone or in combination with pharmaceutically acceptable carriers or diluents, and such administration may be carried out in single or multiple doses.

Compositions may, for example, be in the form of tablets, pills sachets, vials, hard or soft capsules, aqueous or oily suspensions, aqueous or oily solutions, emulsions, powders, granules, syrups, elixirs, lozenges, reconstitutable powders, liquid preparations, creams, troches, hard candies, sprays, creams, salves, suppositories, jellies, gels, pastes, lotions, ointments, injectable solutions, ointments, liquid aerosols, dry powder formulations, HFA aerosols or organic or inorganic acid addition salts.

The compositions of the invention may be in a form suitable for administration through oral, parenteral, subcutaneous, intravenous, intramuscular, buccal, or for administration by inhalation or insufflation (e.g. nasal, tracheal, bronchial) routes.

Depending upon the disorder and patient to be treated and the route of administration, the compositions may be administered at varying doses.

Oral/Buccal/Sublingual

For oral, buccal or sublingual administration, the compounds of the present invention may be combined with various excipients. Solid pharmaceutical preparations for oral administration often include binding agents (for example syrups and sugars, acacia, gelatin, sorbitol, tragacanth, polyvinylpyrrolidone, sodium lauryl sulphate, pregelatinized maize starch, hydroxypropyl methylcellulose, lactose, starches, modified starches, gum acacia, gum tragacanth, guar gum, pectin, wax binders, microcrystalline cellulose, methylcellulose, carboxymethylcellulose, hydroxypropyl methylcellulose, hydroxyethyl cellulose, hydroxypropyl cellulose, copolyvidone and sodium alginate), disintegrants (such as starch and preferably corn, potato or tapioca starch, alginic acid and certain complex silicates, polyvinylpyrrolidone, sucrose, gelatin, acacia, sodium starch glycollate, microcrystalline cellulose, crosscarmellose sodium, crospovidone, hydroxypropyl methylcellulose and hydroxypropyl cellulose), lubricating agents (such as magnesium stearate, sodium lauryl sulfate, talc, silica polyethylene glycol waxes, stearic acid, palmitic acid, calcium stearate, carnuba wax, hydrogenated vegetable oils, mineral oils, polyethylene glycols and sodium stearyl fumarate) and fillers (including high molecular weight polyethylene glycols, lactose, sugar, calcium phosphate, sorbitol, glycine magnesium stearate, starch, glucose, lactose, sucrose, rice flour, chalk, gelatin, microcrystalline cellulose, calcium sulphate, xylitol and lactitol). Such preparations may also include preservative agents and anti-oxidants.

Liquid compositions for oral administration may be in the form of, for example, emulsions, syrups, or elixirs, or may be presented as a dry product for reconstitution with water or other suitable vehicle before use. Such liquid compositions may contain conventional additives such as suspending agents (e.g. sorbitol, syrup, methyl cellulose, hydrogenated edible fats, gelatin, hydroxyalkylcelluloses, carboxymethylcellulose, aluminium stearate gel, hydrogenated edible fats) emulsifying agents (e.g. lecithin, sorbitan monooleate, or acacia), aqueous or non-aqueous vehicles (including edible oils, e.g. almond oil, fractionated coconut oil) oily esters (for example esters of glycerine, propylene glycol, polyethylene glycol or ethyl alcohol), glycerine, water or normal saline; preservatives (e.g. methyl or propyl p-hydroxybenzoate or sorbic acid) and conventional flavouring, preservative, sweetening or colouring agents. Diluents such as water, ethanol, propylene glycol, glycerin and combinations thereof may also be included.

Other suitable fillers, binders, disintegrants, lubricants and additional excipients are well known to a person skilled in the art.

Nasal/Inhalation

For intranasal administration or administration by inhalation, the compounds of the present invention may be delivered in the form of a solution, dry powder or suspension. Administration may take place via a pump spray container that is squeezed or pumped by the patient or through an aerosol spray presentation from a pressurized container or a nebulizer, with the use of a suitable propellant, e.g., dichlorodifluoromethane, trichlorofluoromethane, dichlorotetrafluoroethane, carbon dioxide or other suitable gas. The compounds of the invention may also be administered via a dry powder inhaler, either as a finely divided powder in combination with a carrier substance (e.g. a saccharide) or as microspheres. The inhaler, pump spray or aerosol spray may be single or multi dose. The dosage may be controlled through a valve which delivers a measured amount of active compound.

Parenteral (I.V. and I.M.)

The compounds of the present invention may be formulated in an injectable form in an aqueous or non-aqueous solution, suspension or emulsion in a pharmaceutically acceptable liquid, e.g. sterile water, 1,3-butanediol or a parenterally acceptable oil or a mixture of liquids. The liquid may contain bacteriostatic agents, anti-oxidants or other preservatives, buffers, solutes, thickening agents, wetting agents, suspending agents or other pharmaceutically acceptable additives. It is common that the liquid is isotonic with blood (e.g. through the addition of salts or glucose), and usually has a pH >8. The liquid is dispensed into unit doses in the form of ampoules, disposable injection devices or vials. Alternatively, the formulation is in the form of a concentrate or a dry preparation which can be reconstituted before use to prepare an injectable formulation.

Controlled/Delayed/Prolonged Release Formulation

The compounds of the invention may also be administered in a controlled release formulation. The compounds are released at the required rate to maintain constant pharmacological activity for a desirable period of time. Such dosage forms provide a supply of a drug to the body during a predetermined period of time and thus maintain drug levels in the therapeutic range for longer periods of time than conventional non-controlled formulations. The compounds may also be formulated in controlled release formulations in which release of the active compound is targeted. For example, release of the compound may be limited to a specific region of the digestive system through the pH sensitivity of the formulation. Such formulations are well known to persons skilled in the art.

Liposomes

The active compounds may be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

1. A use of human herpes virus 6 (HHV-6) or derivatives there of in the manufacture of a pharmaceutical composition for the prevention of IgE sensitization, atopy or atopic disease including asthma in children.

2. A use according to claim 1 wherein the pharmaceutical composition comprises live human herpes virus 6 (HHV-6).

3. A use according to claim 1 wherein the pharmaceutical composition comprises weakened human herpes virus 6 (HHV-6).

4. A use according to claim 1 wherein the pharmaceutical composition comprises killed human herpes virus 6 (HHV-6).

5. A use according to claim 1 wherein the pharmaceutical composition comprises one or more fragments derived from human herpes virus 6 (HHV-6).

6. A use according to claim 1 wherein the pharmaceutical composition comprises one or more fragments of human herpes virus 6 (HHV-6) synthesized by recombinant technology.

7. A use according to claim 1 wherein the pharmaceutical composition is to be administered orally.

8. A use according to claim 1 wherein the pharmaceutical composition is to be administered parenterally.

9. A use according to claim 1 wherein the pharmaceutical composition is to be administered onto a mucous membrane.

10. A use according to claim 1 wherein the pharmaceutical composition is to be administered to children within 3 years after birth.

11. A use according to claim 1 wherein the pharmaceutical composition is to be administered to children within 2 years after birth.

12. A use according to claim 1 wherein the pharmaceutical composition is to be administered to children within 18 months after birth.

13. A use according to claim 1 wherein the pharmaceutical composition is to be administered to children within 12 months after birth.

14. A use according to claim 1 wherein the pharmaceutical composition is to be administered to children within 6 months after birth.

15. A use according to claim 1 wherein the pharmaceutical composition is to be administered to children within 4 months after birth.

16. A use according to claim 1 wherein the pharmaceutical composition down-modulates the allergic response to allergens.

17. A use according to claim 1 wherein the pharmaceutical composition inactivates antigen-specific T-cells.

18. A use according to claim 1 wherein the pharmaceutical composition promotes T-cells to secrete cytokines that counteract the development of a Th2 response.

19. A use according to claim 1 wherein the pharmaceutical composition promotes the activation of regulatory T cells.

20. A use according to claim 1 wherein the pharmaceutical composition promotes the secretion of a chemokine from T cells binding the chemokine receptor CCR2 to counteract the development of a Th2 response.

21. A method for preventing IgE-sensitization, allergy, autoimmune disorders and/or inflammatory diseases in humans, by administering a therapeutic effective amount of a human herpes virus 6 (HHV-6) to children no later than 18 months after birth.
